(19) United States
(12) Patent Application Publication
(10) Pub. No.: US 2020/0330186 A1
(43) Pub. Date: Oct. 22, 2020

(54) Laser Guide

(71) Applicant: BOARD OF REGENTS OF THE UNIVERSITY OF TEXAS SYSTEM, Austin, TX (US)

(72) Inventors: Juliana Barros, Houston, TX (US); Shalizeh PATEL, Houston, TX (US); Austin LEDINGHAM, Sheridan, WY (US); Fred Kurtis KASPER, Houston, TX (US)

(21) Appl. No.: 16/853,325

(22) Filed: Apr. 20, 2020

Related U.S. Application Data
(60) Provisional application No. 62/836,837, filed on Apr. 22, 2019.

(51) Int. Cl. A61C 1/08; A61C 19/06; A61N 5/06

(52) U.S. Cl. CPC A61C 1/088; A61C 19/06; A61N 5/0613; A61N 2005/067; A61N 2005/0642; A61N 2005/0644; A61N 2005/0632

(57) ABSTRACT

Disclosed are laser guides for guiding a handheld laser during intra-oral photobiomodulation. The guides include a hollow end cap detachably secured over an end of the laser hand piece, with an opening larger than the laser spot size exiting the handpiece. A hollow conical frustum extends from the end cap and has a lower opening with a diameter sufficiently large so that the guide does not interfere with the laser. A lower base of the frustum may rest on a patient's tissue so as to maintain a predetermined separation distance between the laser exiting the hand piece and the patient's tissue.

### CROSS REFERENCE TO RELATED APPLICATION(S)

This application claims a benefit of priority from U.S. Provisional Application Ser. No. 62/836,837, filed on Apr. 22, 2019, entitled “Laser Guide,” which is fully incorporated by reference herein for all purposes.

### STATEMENT REGARDING FEDERALLY SPONSORED RESEARCH OR DEVELOPMENT

Not applicable.

### TECHNICAL FIELD

This disclosure relates to the field of dentistry, and more particularly to lasers useful for intra-oral photobiomodulation.

### BACKGROUND

Lasers may be employed for numerous purposes in medical and dental applications. For example, in dental application, lasers may be employed for teeth whitening, pain relief, healing, periodontal care, caries removal and many other surgical applications. In particular, diode lasers are used for soft tissue procedures. Such a diode laser used in dental applications may include a fixed laser source and a fiber optic cable used to direct the laser source through a moveable handpiece and toward a patient's mouth, either for intra-oral or extra-oral application of the laser energy.

Applications for lasers in dentistry may require the use of a certain laser power, laser area or spot size, and time. For example, a laser used for surgical cutting may require a higher output power, a smaller laser spot size, and/or a short duration for the application of the laser energy. On the other hand, a laser used to manage oral or maxillofacial pain may require a much lower power, a larger spot size, and a longer duration for the application of the laser energy.

The management of pain using light supplied from a laser is an example of a type of photobiomodulation therapy. Photobiomodulation uses a light source, such as a low-energy laser, to supply photonic energy to affected tissue in order to stimulate positive photochemical changes in light receptive cellular structures, such as mitochondria. Photobiomodulation therapy may include managing pain, reducing inflammation, and enhancing healing or photostimulation.

One difficulty that may arise with these laser therapies is that a single laser source may not be suitable for use in several different applications, due either to the laser power, spot size, or dose requirements. For example, a typical laser used in surgical applications may be effective for cutting, removing tissue, or for coagulating blood, but it may be inappropriate for photobiomodulation treatment. One solution would be to employ several different types of lasers, each suitable to a particular application. Another solution would be to manually move the laser source closer or farther away from the desired target so as to increase or decrease the laser spot size and the applied laser energy density.

A dental laser used in surgical applications may employ a handpiece and a glass fiber tip which focuses the laser energy in a small spot size to the exit of the handpiece. In this manner, the laser energy may be more easily directed toward a very specific target and carefully controlled over the short duration in which laser energy is employed. It may be possible to use such a surgical laser for photobiomodulation therapies by moving the laser source farther away from target, thus increasing the spot size and effectively reducing the power supplied per unit of area. It may be possible to use a surgical laser in this manner for photobiomodulation by manually holding the laser source at the required distance away from the target for the duration required for a particular photobiomodulation therapy. However, this may prove impractical or difficult. By way of example, some photobiomodulation therapies may require a dose of only 30 seconds, while others may require a dose of over 4 minutes, depending on the laser output power and the clinical objectives. Given this requirement to supply a longer dose of laser energy, it may be impossible for a human user to hold a higher power surgical laser at the required location and the required distance for the duration necessary to provide effective therapy. Any human errors in holding the laser at the required location d distance may undesirably either destroy tissue or fail to provide sufficient light for effective photobiomodulation.

### SUMMARY

Disclosed are guides for guiding a handheld. laser during intra-oral photobiomodulation therapy. The guide has a hollow end cap with a longitudinal axis that is fitted over the end of a handpiece of a surgical diode dental laser. The end cap has a proximal end forming a first opening aligned with the longitudinal axis, and a distal end forming a second opening aligned with the axis. The first opening on the end cap is configured to be detachably secured over the end of the handpiece of the laser, and the second opening has a diameter at least as large as the diameter of the laser spot as it exits the handpiece.

A hollow conical frustum extends from the distal end of the end cap. The conical frustum has a longitudinal axis that is also aligned with the longitudinal axis of the end cap. The conical frustum includes an upper base adjacent to the distal end of the end cap. This upper base forms an upper opening having a diameter at least as large as the diameter of the laser spot exiting the handpiece, such that the laser guide does not interfere with the laser exiting the handpiece.

The conical frustum further includes a lower base spaced apart from the upper base and terminating at a lower opening. This lower opening has a diameter larger than the diameter of the upper opening and larger than the diameter of the laser spot exiting the lower opening. In this manner, the lower base may be positioned on a patient's intra-oral tissue and function to maintain a predetermined separation distance between the laser exiting the handpiece and the target of the laser. This predetermined separation distance maintains a larger laser spot size and may permit the use of a surgical laser for intra-oral photobiomodulation therapy.

### BRIEF DESCRIPTION OF THE DRAWINGS

Hereinafter, examples of laser guides will be described with reference to the drawings.

FIG. 1 is a perspective view of a dental laser.

FIG. 2 is a side view of a hand piece of a dental laser and a focused laser beam emitted therefrom.

FIG. 3 is a perspective view of an example of a laser guide.

FIG. 4 is side view of a handpiece of a dental laser with an example of a laser guide attached thereto.

### DETAILED DESCRIPTION

Examples of laser guides and their various features are now explained more fully with reference to certain non-limiting features that are illustrated in the accompanying drawings and detailed in the following description. Descriptions of well-known materials, manufacturing techniques, parts, and equipment are omitted. It should be understood, however, that the detailed description and the specific examples, while indicating preferred examples or embodiments, are given by way of illustration only and not by way of limitation. Various substitutions, modifications, additions and/or rearrangements within the spirit and/or scope of the underlying concepts will become apparent to those skilled in the art from this disclosure.

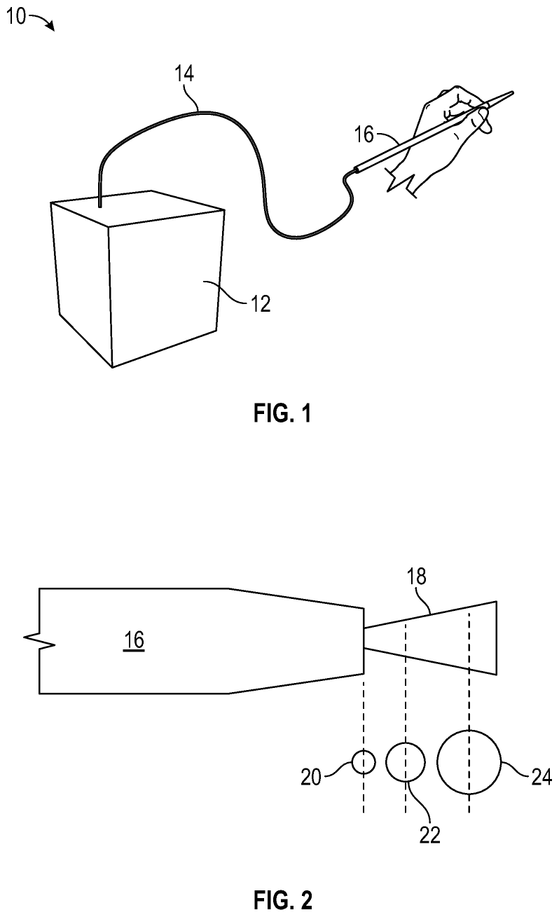

FIGS. 1 and 2 illustrate a dental laser 10. Dental laser 10 includes a laser source 12 producing a laser output. The laser output is directed through a fiber optic cable 14 which extends from the laser source 12 and travels through a handpiece 16 positioned at the distal end of the fiber optic cable 14. A user may grip the handpiece 16 and direct the laser exiting the end of the fiber optic cable.

A dental laser 10 used for surgical procedures may be configured such that the laser beam is focused and has narrow spot diameter 20 at the exit of hand piece 16. As the separation distance from the exit of the handpiece 16 increases, the spot diameter of the laser beam increases, illustrated by laser spots 22 and 24 with increasing diameters, as shown in FIG. 2.

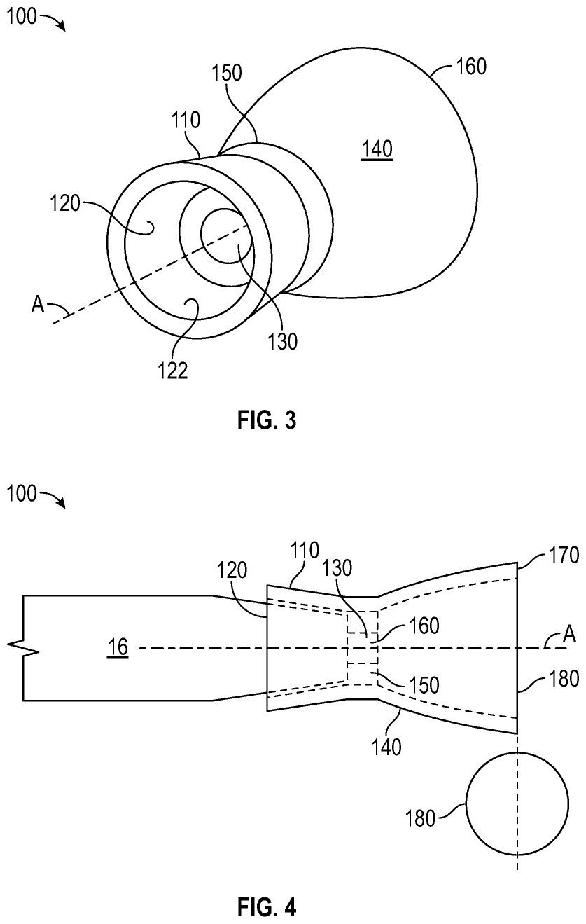

A laser guide 100 is illustrated in FIGS. 3 and 4. The laser guide 100 is removably attached to the end of handpiece 16. Laser guide 100 generally includes a hollow end cap 110 that may be fitted over the end of handpiece 16. By way of example, the laser guide 100 may be formed of an elastic material and may be secured by friction over the end of the handpiece 16. The laser guide 100 may also include threads (not shown) on the interior surface 122 of the end cap 110 so as to form a threaded connection with the end of the handpiece 16. While not shown in the drawings, the end cap 110 may also be secured by other means, such as a bayonet connection or protrusions to engage with indentations in the handpiece 16.

Laser guide 100 may be formed in one piece by injection molding an elastic material. Laser guide 100 may also be formed from a transparent or semi-transparent material so that a laser spot size remains at least partly visible when viewed through the laser guide by an operator during operation of the laser 10.

Laser guide has a longitudinal axis A aligned with the center of the laser beam exiting the handpiece 16. The end cap 110 has a proximal end that forms a first opening 120 aligned with the axis A, and a distal end forming a second opening 130, which is also aligned with the axis A. The first opening 120 is secured over the end of the handpiece 16 and the second opening 130 forms a diameter at least as large as the diameter 20 of the laser spot as it exits the handpiece 16 (as shown in FIG. 2).

A hollow conical frustum 140 extends from the distal end of the end cap 110 in order to provide a physical guide separating the end of the handpiece 16 from the patient's intra-oral tissue. The conical frustum 140 is also aligned with the longitudinal axis A and includes an upper base 150 adjacent to the distal end of the end cap 110 and a lower base 170 which may touch or rest on the patient's tissue during operation of the laser 10. The upper base 150 forms an upper opening 160 that has a diameter that is also at least as large as the diameter of the laser spot exiting the hand piece 16.

The lower base 170 of the conical frustum 140 terminates at a lower opening 180. Lower opening 180 has a diameter larger than the diameter of the upper opening 160 and larger than the diameter of the laser spot exiting the lower opening 180, so that the conical frustum 140 does not disturb the laser beam during operation. For example, lower opening 180 may have a larger diameter than the diameter 22 or diameter 24 as shown in FIG. 2.

During use, the lower base 170 of the laser guide 100 may be held or rest against a target tissue in the patient's mouth. The separation provided by the laser guide 100 provides a larger laser spot diameter at the patient's tissue, which is generally desirable for photobiomodulation applications. In this manner, the laser guide 100 also relieves strain on the user who must otherwise manually maintain the laser handpiece 16 at a short distance from the patient's tissue for longer time durations.

As shown in FIG. 2, the spot diameter of the laser beam generally increases with distance away from the tip of the handpiece 16. As the spot diameter of the laser beam increases, the effective energy density of the laser decreases. For surgical applications, a narrower spot diameter for increased precision and a higher energy density is generally desirable to quickly cut or remove tissue. For example, the average power needed to start excising any tissue with diode lasers is 1.0 W or higher. For non-surgical applications, a small laser spot size is generally not desirable. Thus, for a given laser power, the desired laser spot size necessary to produce coagulation is generally larger than the spot size useful for cutting or vaporizing tissue. In turn, the desired laser spot size necessary to produce photostimulation effects is generally larger and will have a low energy density. For example, the average power needed for photobiomodulation ranges from 0.5 w to 0.8 W.

As noted, photobiomodulation may also require the application of laser energy for a much longer duration in a static position, different from the laser energy applied to quickly vaporize tissue in surgical applications. For example, photobiomodulation therapies may require a dose that can vary from 30 seconds to over 4 minutes. This requirement to supply a longer dose of laser energy generally also requires the use of either a lower power laser and/or a much larger laser spot size. Using excessive power or a too small spot size may produce undesirable thermal effects and/or tissue damage.

The relationship between the time that a laser is applied to tissue in order to produce a given effect and the laser intensity, laser spot size, and laser power may be represented by the following formula:

T(s)=ED×A/P

where the time (T) in seconds is equal to the energy density (ED) in joules per square centimeter multiplied by the laser spot area (A) in square centimeters, divided by the output power (P) in watts. Because the area of a circular laser spot is proportional to the square of the spot diameter, it can be seen that the time required for a given effect is also proportional to the square of the spot diameter.

The disclosed laser guides 100 effectively stabilize the laser spot emanating from the end of handpiece 16 at a separation distance from the target tissue sufficient to provide a larger spot diameter, thereby allowing an appropriate laser dose that is generally necessary for photobiomodulation. Without a laser guide, it may prove difficult or impossible for a human user to hold a laser at the required distance for the duration necessary to provide effective photobiomodulation. Human errors in holding the laser at the required distance may undesirably fail to accurately and repeatedly deliver the dose needed to achieve the intended clinical effects.

By way of example, a surgical diode laser was tested to determine a suitable separation distance between the end of the handpiece and target tissue in order to produce a sufficiently larger laser spot diameter and provide effective photobiomodulation therapy. The diode laser tested has a wavelength of 940 nm, at 600 mW output power. This device was tested without the surgical glass fiber tip the end of the handpiece. As the separation distance between the end of the handpiece and the patient's tissue increased, the laser spot diameter also increased. The time required for effective photobiomodulation was computed using five predetermined separation distances of 0, 15, 20, 25, and 30 mm.

Among the five pre-determined separation distances tested, a shorter distance of 15 mm provided a laser spot diameter of about 15 mm, and thereby allowed an effective dose delivery for many different intra-oral photobiomodulation applications. Using this separation distance of 15 mm, photobiostimulation required a laser irradiation of 52 seconds. For anti-inflammatory and analgesic effects, the time of each treatment increased to 130 seconds and 262 seconds, respectively.

Accordingly, for the laser utilized during testing, a laser guide 100 having a separation distance of 15 mm may be effective for several different photobiomodulation therapies. A like calculation may be utilized to choose a separation distance useful for other dental lasers used for photobiomodulation. Once a desired separation distance is determined, a laser guide 100 may be selected to provide the desired separation distance. In the examples described above, the separation distance is the distance between the lower opening 130 of the end cap 110 and the lower base 170 of the conical frustum 140.

For these reasons, the laser guide 100 may be manufactured in several different sizes adapted to provide a predetermined separation distance effective for photobiomodulation therapy using different model dental lasers. The laser guide 100 may also be inexpensively manufactured so that it may be disposed after a single use.

It is to be noted that various modifications or alterations can be made to the above-described examples without departing from the technical features of the inventions as defined in the appended claims.

What is claimed is:

1. An apparatus for guiding a handheld laser during intra-oral photobiomodulation, the apparatus comprising:
a hollow end cap having a longitudinal axis, a proximal end forming a first opening aligned with the longitudinal axis, and a distal end forming a second opening aligned with the axis, wherein the first opening is configured to be detachably secured over an end of a hand piece of the laser and the second opening has a diameter at least as large as a diameter of a laser spot exiting the handpiece; and
a hollow conical frustum extending from the distal end of the end cap and having a longitudinal axis aligned with the longitudinal axis of the end cap, the frustum comprising:
an upper base adjacent to the distal end of the end cap and forming an upper opening having a diameter at least as large as the diameter of the laser spot exiting the hand piece; and
a lower base spaced apart from the upper base and terminating at a lower opening having a diameter larger than the diameter of the upper opening and larger than a diameter of the laser spot exiting the lower opening,
such that the lower base functions to maintain a predetermined separation distance between the laser exiting the hand piece and an intra-oral target of the laser.
